(19) United States
(12) Patent Application Publication
(10) Pub. No.: US 2024/0386657 A1
(43) Pub. Date: Nov. 21, 2024

(54) Physical World Driven Environmental Themes for Avatars in Virtual/Augmented Reality Systems

(71) Applicant: International Business Machines Corporation, Armonk, NY (US)

(72) Inventors: Peng Hui Jiang, Beijing (CN); Jun Su, Beijing (CN); Su Liu, Austin, TX (US); Yu Zhu, Beijing (CN); Guang Han Sui, Beijing (CN)

(21) Appl. No.: 18/197,327

(22) Filed: May 15, 2023

(51) Int. Cl. G06T 17/00; A61B 5/16; G06V 40/16

(52) U.S. Cl. CPC G06T 17/00; A61B 5/165; G06V 40/174

(57) ABSTRACT

Mechanisms are provided for personalizing a computer generated virtual environment. Sensors associated with a user collect emotion data representing physiological conditions of the user in response to stimuli. Source computing systems collect stimuli context data and the stimuli context data is correlated with the emotion data. Machine learning model(s) are trained, based on the emotion data and correlated stimuli context data, to predict an emotion of the user from patterns of input data. Runtime emotion data is received from the sensors, and runtime stimuli context data is received from a virtual environment provider computing system for a computer generated virtual environment. The trained machine learning model(s) generate a predicted emotion of the user based on the runtime emotion data and the runtime stimuli context data. In cases, the virtual environment is modified based on the predicted emotion of the user.

### BACKGROUND

The present application relates generally to an improved data processing apparatus and method and more specifically to an improved computing tool and improved computing tool operations/functionality for physical world driven environmental themes for avatars in virtual/augmented reality systems.

Virtual reality environments are an area of interest to many individuals and large technology oriented organizations because they provide a space through which users may interact, even if such users may be physically located remote from each other in the physical world. Virtual reality environments provide a virtual space in which users can interact with computer-generated environments and other users. Virtual reality environments have often been utilized in video gaming and social networking applications, such as massively multiplayer online (MMO) games, e.g., World of Warcraft® available from Blizzard Entertainment, Inc. of Los Angeles, California, or Second Life® world, available from Linden Lab® of San Francisco, California.

The Metaverse has recently been given much attention as a network of interoperable virtual worlds including augmented reality (AR) and virtual reality (VR) through which users may collaborate, shop, explore, and play games. Typically, a user is represented in the virtual reality space by an avatar, which is a digital representation of the user, but which does not need to resemble the user and can be any desirable representation the user chooses within the confines of what is made available in the virtual reality computer application. Users, via their avatars, are able to shop for virtual products, including unique items protected by non-fungible tokens (NFTs), play in sporting events of virtualized sports leagues, attend virtualized musical events, such as concerts and the like, attend work meetings, collaborate with co-workers on projects, etc. The Metaverse is intended to be an ever present, and ever evolving virtual space through which users may experience a virtualized life.

### SUMMARY

This Summary is provided to introduce a selection of concepts in a simplified form that are further described herein in the Detailed Description. This Summary is not intended to identify key factors or essential features of the claimed subject matter, nor is it intended to be used to limit the scope of the claimed subject matter.

In one illustrative embodiment, a method, in a data processing system, is provided for personalizing a computer generated virtual environment. The method comprises collecting, from one or more sensors associated with a user, emotion data representing physiological conditions of the user in response to stimuli, and collecting, from one or more data source computing systems, stimuli context data and correlating the stimuli context data with the emotion data. The method also comprises training, via a machine learning training process, one or more machine learning computer models based on the emotion data and correlated stimuli context data to thereby generate one or more trained machine learning computer models that are trained to predict an emotion of the user from patterns of input data. The method further comprises receiving runtime emotion data from the one or more sensors associated with the user, and receiving runtime stimuli context data from a virtual environment provider computing system for the computer generated virtual environment. In addition, the method comprises generating, by the one or more trained machine learning computer models, a predicted emotion of the user based on the runtime emotion data and the runtime stimuli context data which are input to the one or more trained machine learning computer models.

In other illustrative embodiments, a computer program product comprising a computer useable or readable medium having a computer readable program is provided. The computer readable program, when executed on a computing device, causes the computing device to perform various ones of, and combinations of, the operations outlined above with regard to the method illustrative embodiment.

In yet another illustrative embodiment, a system/apparatus is provided. The system/apparatus may comprise one or more processors and a memory coupled to the one or more processors. The memory may comprise instructions which, when executed by the one or more processors, cause the one or more processors to perform various ones of, and combinations of, the operations outlined above with regard to the method illustrative embodiment.

These and other features and advantages of the present invention will be described in, or will become apparent to those of ordinary skill in the art in view of, the following detailed description of the example embodiments of the present invention.

### BRIEF DESCRIPTION OF THE DRAWINGS

The invention, as well as a preferred mode of use and further objectives and advantages thereof, will best be understood by reference to the following detailed description of illustrative embodiments when read in conjunction with the accompanying drawings, wherein:

FIG. 1 is an example diagram of a distributed data processing system environment in which aspects of the illustrative embodiments may be implemented and at least some of the computer code involved in performing the inventive methods may be executed;

FIG. 2 is an example block diagram illustrating the primary operational components of an augmented environmental theme for avatars (AETA) computing system in accordance with one illustrative embodiment;

FIG. 3 is an example data flow diagram for an AETA computing system in accordance with one illustrative embodiment;

FIG. 4 is a flowchart outlining an example operation of an AETA computing system in accordance with one illustrative embodiment;

FIG. 5 is a flowchart outlining an example operation of an emotion classifier/predictor engine of an AETA computing system in accordance with one illustrative embodiment; and

FIG. 6 is a flowchart outlining an example operation of a virtual environment personalization engine of an AETA computing system in accordance with one illustrative embodiment.

### DETAILED DESCRIPTION

As noted above, virtual/augmented reality computing systems are being proliferated in the current information technology and computer oriented society as a mechanism to facilitate interactions between users for work and entertainment purposes. However, there are still many limits to the adequacy of computing systems to provide such virtual/augmented reality environments in a manner that accurately represents users in the virtual environment through their avatars. For example, current virtual/augmented reality environments do not provide adequate representation of a user's emotional dispositions and responses towards environmental features and/or other users. Moreover, current virtual/augmented reality environments do not provide adequate add-on services and/or virtual content based on emotional characteristic indicators of users to enhance the user's experience within the virtual/augmented reality environment.

The illustrative embodiments provide an improved computing tool and improved computing tool functionality/operations specifically directed to improving the manner by which virtual/augmented reality environments, and user avatars within the virtual/augmented reality environments, are rendered so as to enhance user experience within the virtual/augmented environment. Specifically, the illustrative embodiments provide improved computing tools and improved computing tool functionality/operations to provide a more immersive and personalized experience within the virtual/augmented environment based on emotional characteristic indicators determined through machine learning model learning of the specific user's emotional disposition and response to various stimuli. The one or more machine learning models are trained to predict the user's current emotional state and changes to that emotional state in response to potential stimuli. The predictions are then used to drive the content of virtual environments and the representation of the virtual environments to the perspective of the user's avatar. This is referred to herein as the environmental “theme” of the avatar. Thus, by learning the emotional states of the user over time with regard to various stimuli, e.g., locations, events, other users, other objects and/or entities, etc., and using the learned associations to predict current and potential emotional status of the user, the virtual environment may be represented to the user's avatar in a personalized manner to elicit a desired emotional state, or otherwise respond to the user's current predicted emotional state. This may be done so as to provide a more immersive experience as well as otherwise improve the user's experience within the virtual environment.

For example, sensor data may be collected from the user while the user is being stimulated by a particular animal present within the user's physical environment, e.g., a dog barking, presence of a cat, a spider or snake moving, or the like. The sensor data may be labeled by the user to specify the stimuli and the emotional state, e.g., spider/afraid. This information may be used as training data that is used, along with other training data, to train a machine learning model to correlate or associate the pattern of sensor data representative of the emotional state of “afraid” with the stimuli of a dog present in the environment. Thereafter, having learned this association, if a virtual environment includes a virtual representation of a spider, it may be predicted that the user will respond to this stimuli with an “afraid” emotional response. As a result, the computing system may modify the virtual environment to remove the virtual spider or replace the virtual spider with a replacement object/entity that will not generate a negative emotional response from the user, or which may generate a targeted emotional response that is desired from the user. It should be appreciated that in some cases, the targeted emotional response may in fact be a negative emotional response, e.g., in some cases it may be desirable to evoke an emotional response of “afraid” or the like, so as to improve the immersive nature of the virtual environment, e.g., if the user's avatar is entering a location within the virtual environment where the user should be afraid, then stimuli that evoke the desired emotional response may be included in the virtual environment.

Thus, the virtual environment may be adapted or modified to the specific personalized emotional responsiveness or disposition of the user to particular stimuli so as to avoid unwanted, or evoke desired, emotional responses from the user and thereby provide a personalized and more immersive experience for the user. It should be appreciated that the illustrative embodiments may operate with regard to various stimuli and emotional states. The stimuli may be anything that may be present within the physical environment of the user, and which may also be represented within the virtual environment, which triggers an emotion in the user, and which can be represented as input data that can be correlated with sensor data indicative of the emotion of the user when presented with the stimuli. Thus, this may include objects, entities, or even characteristics of object/entities, e.g., particular colors, particular tonalities of sounds and/or voices, and the like. For example, visual stimuli may be colors, shapes, textures, facial expressions, body language, and the like. Auditory stimuli may be music, sounds, voices, and the like. Environmental stimuli may be temperature, lighting, weather, and the like. Social stimuli may be other users, interactions with other avatars, memories, past experiences, and personal preferences. Object stimuli may be animals, vehicles, and other objects that are present in the environment. Contextual stimuli may be the user's location, time of day, current task, and the like.

A stimuli ontology data structure that provides pre-defined stimuli and their characteristics may be used to represent these recognizable stimuli, e.g., an entry in the stimuli ontology data structure for “spider” may include characteristics such as “type: arachnid”, “number of legs: 8”, “mode of movement: crawling”, “number of eyes: many”, “size: small to medium”, etc. The same is true of the emotional states of users, i.e., an emotional state ontology data structure may specify the recognizable emotional states and their characteristics so as to allow for correlating characteristics of emotional states with characteristics of stimuli. For example, an emotional state of “afraid” may have characteristics of “type: negative”, “heart rate: X”, “brain waves: Y”, etc. The ontology data structures may initially be generalized, but then instances of the ontology data structures may be customized to the particular users based on the detected emotional states/stimuli and the learned associations between these. In addition, additional entries may be added to the ontology data structures based on the sensor data collected for various stimuli and inputs from the user specifying the stimuli and corresponding emotional state.

The learned ontology data structures may be utilized as inputs to train the machine learning models for the particular users. The resulting trained machine learning models may then be applied to runtime data to predict current/future emotional states of the user given particular stimuli. The particular stimuli may be, for example, elements of the virtual environment that the user's avatar is currently in or is likely to move to within the virtual environment. The predictions may then be used to modify the virtual environment by modifying the environment theme to elicit a desired emotional response, which may be positive or negative to the particular user. It should be appreciated that this change in virtual environment theme may only be perceived by the user via their user avatar and may not result in a change to other user's experience of the virtual environment. That is, if multiple users' avatars are in the same location of a virtual environment, each may have a different experience and be presented with different environment themes within the virtual environment for the same location, depending on their particular associations of emotional state and stimuli, e.g., one user may be afraid of spiders while another is not, so the first user may be presented with spiders to elicit the emotional state of “afraid” while the other is presented with a different stimuli, e.g., snakes, to elicit the same emotional state of “afraid”. Thus, the virtual environment theme may be personalized to the particular learned associations of emotional state and stimuli. This results in a more personalize and immersive virtual environment experience for each user.

It should be appreciated that while the description of the illustrative embodiments will make reference to virtual environments, the illustrative embodiments may be implemented with regard to any fully or partially-represented virtual environment. A fully virtual environment is one in which the environment of the user, as a whole, is completely represented by a computer generated environment in which the user is represented as an avatar or the user's viewpoint is from that of an avatar that fully exists within the computer generated environment. A partially-represented virtual environment is one in which some aspects of the physical (real) world are still perceivable while also perceiving some aspects of a virtual world, e.g., augmented reality environments in which the physical (real) world environment may serve as a background upon which virtual reality environment features are also represented, such as virtual objects represented as being present within the physical world, although still being virtual. In addition, while the illustrative embodiments will be described with regard to a single virtual environment for ease of explanation, the illustrative embodiments may be implemented across multiple virtual/augmented environments, such as in the case of the Metaverse, for example.

It should also be appreciated that the description of the illustrative embodiments will make reference to one or more machine learning models which are implemented as computer models that are trained through machine learning processes based on training datasets, testing using testing datasets, and then operating on runtime acquired data to make classifications/predictions based on learned associations between patterns of input data and resulting classifications/predictions. The machine learning models may comprise one or more machine learning models. Moreover, where multiple machine learning models are represented, it should be appreciated that all of these machine learning models, or subsets of these machine learning models, may be combined into a single machine learning model, where appropriate to the desired implementation. In addition, in some illustrative embodiments, multiple machine learning computer models may be configured into a machine learning model or artificial intelligence (AI) computer pipeline in which the output of one or more machine learning models may serve as input to other downstream machine learning model(s) of the pipeline. In some illustrative embodiments the machine learning computer models may be implemented as ensembles of machine learning computer models in which the outputs of multiple machine learning computer models are combined, potentially by a meta-machine learning computer model, e.g., meta-classifier or meta-prediction model, to generate a combined classification/prediction.

Overall, the illustrative embodiments provide an improved artificial intelligence (AI) computer system comprising a plurality of specifically configured and trained AI computer tools, e.g., neural networks, deep learning computer models, cognitive computing systems, or other AI mechanisms that are trained based on a finite set of data to perform specific tasks. The configured and trained AI computer tools are each specifically configured/trained to perform a specific type of artificial intelligence processing of inputs, represented as one or more collections of data and/or metadata that define sensor data indicative of an emotional state of a user and/or stimuli associated with the emotional state of the user. In general, these AI tools employ machine learning (ML)/deep learning (DL) computer models (or simply ML models) to perform tasks that, while emulating human thought processes with regard to the results generated, use different computer processes, specific to computer tools and specifically ML/DL computer models, which learn patterns and relationships between data that are representative of particular results, e.g., relationships between patterns of input data representing sensor data and stimuli, and corresponding emotional state classifications/predictions. The ML/DL computer model is essentially a function of elements including the machine learning algorithm(s), configuration settings of the machine learning algorithm(s), features of input data identified by the ML/DL computer model, and the labels (or outputs) generated by the ML/DL computer model, where these labels represent classifications or predictions based on the patterns recognized in the inputs to the ML/DL computer model. By specifically tuning the function of these elements through a machine learning process, a specific ML/DL computer model instance is generated. Different ML models may be specifically configured and trained to perform different AI functions with regard to the same or different input data.

As the artificial intelligence (AI) computer system implements a plurality of ML/DL computer models, it should be appreciated that these ML/DL computer models are trained through ML/DL processes for specific purposes. Example machine learning techniques that may be used to construct and train such ML/DL computer models may include, but are not limited to, nearest neighbor (NN) techniques (e.g., k-NN models, replicator NN models, etc.), statistical techniques (e.g., Bayesian networks, etc.), clustering techniques (e.g., k-means, etc.), neural networks (e.g., reservoir networks, artificial neural networks, etc.), support vector machines (SVMs), or the like.

Thus, as an overview of the ML/DL computer model training processes, it should be appreciated that machine learning is concerned with the design and the development of techniques that take as input empirical data (such as sensor data, location data, event data, virtual environment location/event data, etc.), and recognizes complex patterns in the input data. One common pattern among machine learning techniques is the use of an underlying computer model M, whose parameters are optimized for minimizing the cost function associated to M, given the input data. For instance, in the context of classification, the model M may be a straight line that separates the data into two classes (e.g., labels) such that M=a*x+b*y+c and the cost function would be the number of misclassified points. The learning process then operates by adjusting the parameters a, b, c such that the number of misclassified points is minimal. After this optimization phase (or learning phase), the model M can be used to classify new data points. Often, M is a statistical model, and the cost function is inversely proportional to the likelihood of M, given the input data. This is just a simple example to provide a general explanation of machine learning training and other types of machine learning using different patterns, cost (or loss) functions, and optimizations, which may be of a more complex nature, may be used with the mechanisms of the illustrative embodiments without departing from the spirit and scope of the present invention.

The processor-implemented artificial intelligence (AI) computer system of the illustrative embodiments generally includes one or both of machine learning (ML) and deep learning (DL) computer models. In some instances, one or the other of ML and DL can be used or implemented to achieve a particular result. Traditional machine learning can include or use algorithms such as Bayes Decision, Regression, Decision Trees/Forests, Support Vector Machines, or Neural Networks, among others. Deep learning can be based on deep neural networks and can use multiple layers, such as convolution layers. Such DL, such as using layered networks, can be efficient in their implementation and can provide enhanced accuracy relative to traditional ML techniques. Traditional ML can be distinguished from DL in general in that DL models can outperform classical ML models, however, DL models can consume a relatively larger amount of processing and/or power resources. In the context of the illustrative embodiments, references herein to one or the other of ML and DL can be understood to encompass one or both forms of AI processing.

Before continuing the discussion of the various aspects of the illustrative embodiments and the improved computer operations performed by the illustrative embodiments, it should first be appreciated that throughout this description the term “mechanism” will be used to refer to elements of the present invention that perform various operations, functions, and the like. A “mechanism,” as the term is used herein, may be an implementation of the functions or aspects of the illustrative embodiments in the form of an apparatus, a procedure, or a computer program product. In the case of a procedure, the procedure is implemented by one or more devices, apparatus, computers, data processing systems, or the like. In the case of a computer program product, the logic represented by computer code or instructions embodied in or on the computer program product is executed by one or more hardware devices in order to implement the functionality or perform the operations associated with the specific “mechanism.” Thus, the mechanisms described herein may be implemented as specialized hardware, software executing on hardware to thereby configure the hardware to implement the specialized functionality of the present invention which the hardware would not otherwise be able to perform, software instructions stored on a medium such that the instructions are readily executable by hardware to thereby specifically configure the hardware to perform the recited functionality and specific computer operations described herein, a procedure or method for executing the functions, or a combination of any of the above.

The present description and claims may make use of the terms “a”, “at least one of”, and “one or more of” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

Moreover, it should be appreciated that the use of the term “engine,” if used herein with regard to describing embodiments and features of the invention, is not intended to be limiting of any particular technological implementation for accomplishing and/or performing the actions, steps, processes, etc., attributable to and/or performed by the engine, but is limited in that the “engine” is implemented in computer technology and its actions, steps, processes, etc. are not performed as mental processes or performed through manual effort, even if the engine may work in conjunction with manual input or may provide output intended for manual or mental consumption. The engine is implemented as one or more of software executing on hardware, dedicated hardware, and/or firmware, or any combination thereof, that is specifically configured to perform the specified functions. The hardware may include, but is not limited to, use of a processor in combination with appropriate software loaded or stored in a machine readable memory and executed by the processor to thereby specifically configure the processor for a specialized purpose that comprises one or more of the functions of one or more embodiments of the present invention. Further, any name associated with a particular engine is, unless otherwise specified, for purposes of convenience of reference and not intended to be limiting to a specific implementation. Additionally, any functionality attributed to an engine may be equally performed by multiple engines, incorporated into and/or combined with the functionality of another engine of the same or different type, or distributed across one or more engines of various configurations.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the examples provided herein without departing from the spirit and scope of the present invention.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

It should be appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

The present invention may be a specifically configured computing system, configured with hardware and/or software that is itself specifically configured to implement the particular mechanisms and functionality described herein, a method implemented by the specifically configured computing system, and/or a computer program product comprising software logic that is loaded into a computing system to specifically configure the computing system to implement the mechanisms and functionality described herein. Whether recited as a system, method, of computer program product, it should be appreciated that the illustrative embodiments described herein are specifically directed to an improved computing tool and the methodology implemented by this improved computing tool. In particular, the improved computing tool of the illustrative embodiments specifically provides machine learning training logic and machine learning training models that learn associations between various stimuli and user emotional states, and then uses these learned associations to predict current/future emotional states of the user which drive representations of virtual environments from the viewpoint of the user's avatar. The improved computing tool implements mechanism and functionality, such as the augmented environmental themes for avatars (AETA) computing system, the machine learning models that are implemented in the AETA computing system, and the virtual environment theme selection and modification mechanisms of the AETA computing system, which cannot be practically performed by human beings either outside of, or with the assistance of, a technical environment, such as a mental process or the like. The improved computing tool provides a practical application of the methodology at least in that the improved computing tool is able to improve user experiences of virtual environments by personalizing the virtual environment representation to the emotional states of the user which improves the immersion of the user into the virtual environment and allows for targeted representations to elicit desired emotional responses by the user.

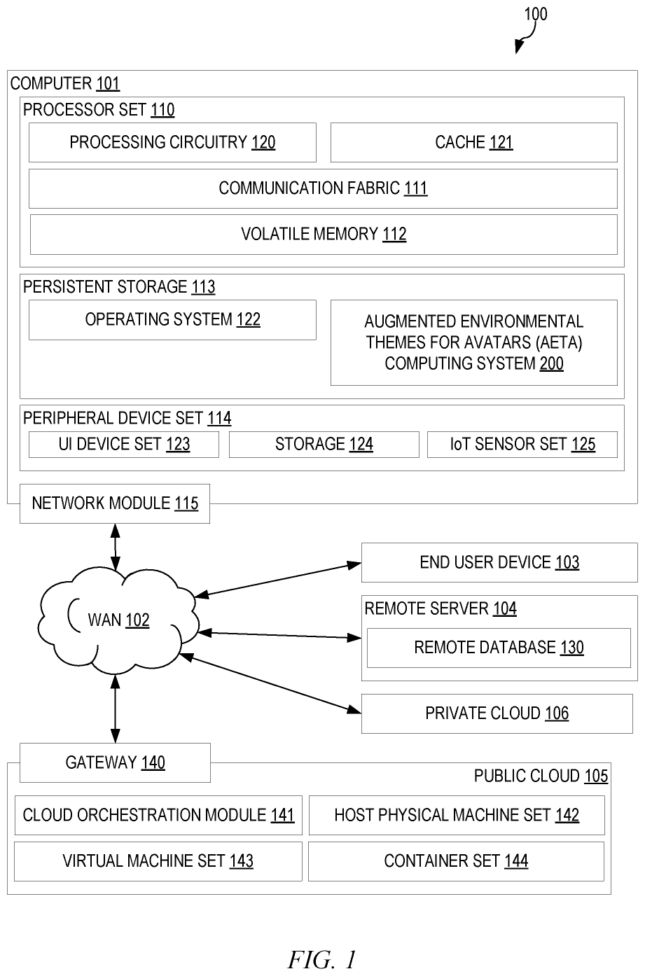

FIG. 1 is an example diagram of a distributed data processing system environment in which aspects of the illustrative embodiments may be implemented and at least some of the computer code involved in performing the inventive methods may be executed. That is, computing environment 100 contains an example of an environment for the execution of at least some of the computer code involved in performing the inventive methods, such as augmented environmental themes for avatars (AETA) computing system 200, which will be described in greater detail hereafter. In addition to AETA computing system 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and AETA computing system 200, as identified above), peripheral device set 114 (including user interface (UI), device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

Computer 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

Processor set 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in AETA computing system 200 in persistent storage 113.

Communication fabric 111 is the signal conduction paths that allow the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

Volatile memory 112 is any type of volatile memory now known or to be developed in the future. Examples include dynamic type random access memory (RAM) or static type RAM. Typically, the volatile memory is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

Persistent storage 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface type operating systems that employ a kernel. The code included in AETA computing system 200 typically includes at least some of the computer code involved in performing the inventive methods.

Peripheral device set 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion type connections (for example, secure digital (SD) card), connections made though local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

Network module 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

End user device (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

Remote server 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

Public cloud 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

Private cloud 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

As shown in FIG. 1, one or more of the computing devices, e.g., computer 101 or remote server 104, may be specifically configured to implement an augmented environmental themes for avatars (AETA) computer system that interacts with one or more virtual/augmented reality environment computing systems to personalize the virtual/augmented reality environment to the specific emotional states and emotional disposition/response of users to particular stimuli. The configuring of the computing device may comprise the providing of application specific hardware, firmware, or the like to facilitate the performance of the operations and generation of the outputs described herein with regard to the illustrative embodiments. The configuring of the computing device may also, or alternatively, comprise the providing of software applications stored in one or more storage devices and loaded into memory of a computing device, such as computer 101 or remote server 104, for causing one or more hardware processors of the computing device to execute the software applications that configure the processors to perform the operations and generate the outputs described herein with regard to the illustrative embodiments. Moreover, any combination of application specific hardware, firmware, software applications executed on hardware, or the like, may be used without departing from the spirit and scope of the illustrative embodiments.

It should be appreciated that once the computing device is configured in one of these ways, the computing device becomes a specialized computing device specifically configured to implement the mechanisms of the illustrative embodiments and is not a general purpose computing device. Moreover, as described hereafter, the implementation of the mechanisms of the illustrative embodiments improves the functionality of the computing device and provides a useful and concrete result that facilitates personalization of virtual/augmented reality environments based on predicted current/future emotional states of a user given one or more stimuli.

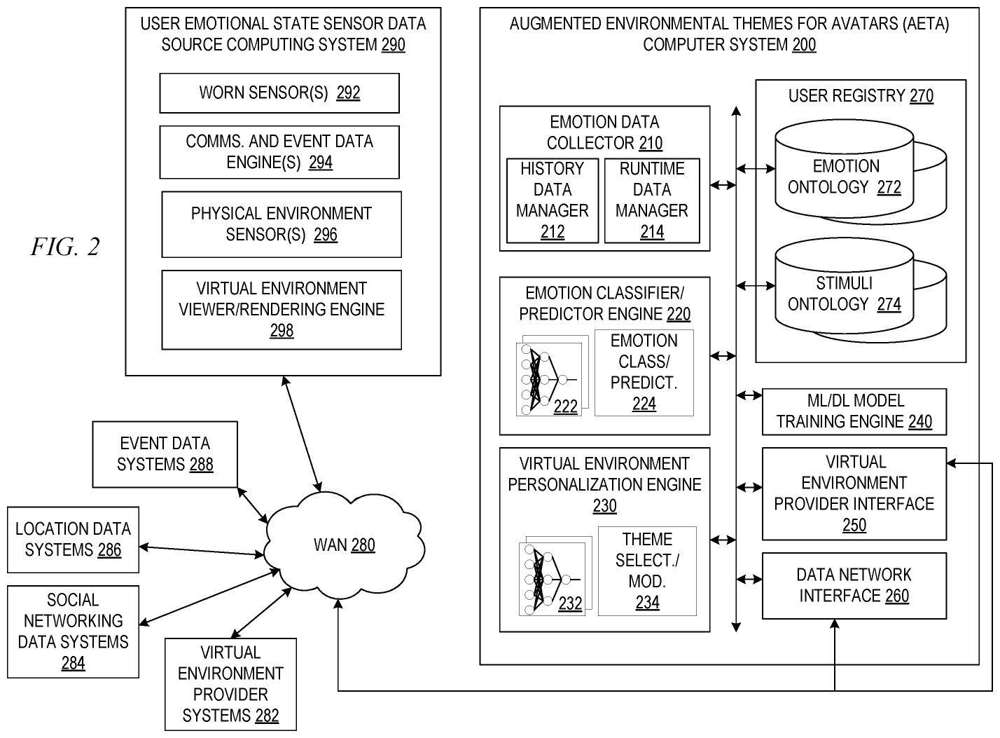

FIG. 2 is an example block diagram illustrating the primary operational components of an augmented environmental theme for avatars (AETA) computing system in accordance with one illustrative embodiment. The operational components shown in FIG. 2 may be implemented as dedicated computer hardware components, computer software executing on computer hardware which is then configured to perform the specific computer operations attributed to that component, or any combination of dedicated computer hardware and computer software configured computer hardware. It should be appreciated that these operational components perform the attributed operations automatically, without human intervention, even though inputs may be provided by human beings, e.g., inputs indicative of emotional state and labeling of stimuli, and the resulting output may aid human beings, e.g., generating a more immersive and personalized experience within a virtual environment. The invention is specifically directed to the automatically operating computer components directed to improving the way that virtual environments are rendered for specific users, and more specifically based on the user's predicted current emotional state and/or potential emotional state given specific stimuli.

The invention provides a specific solution that implements machine learning training of machine learning models specifically with regard to users' emotional states in response to stimuli and using the learned associations to predict current and/or potential emotional states of the user so as to personalize a virtual environment to the user to improve the user's experience within the virtual environment. As the invention is specifically directed to virtual environments, adapting or modifying the virtual environments to the emotional states of the user, and implementing machine learning training and machine learning models to perform such operations, the improved computing tools and improved computing tool functionality of the present invention cannot be practically performed by human beings as a mental process and are not directed to organizing any human activity.

As shown in FIG. 2, the augmented environmental themes for avatars (AETA) computing system 200 comprises an emotion data collector engine (emotion data collector) 210, an emotion classifier/predictor engine 220, a virtual environment personalization engine 230, a user registry 270, a machine learning/deep learning model training engine 240, a virtual environment provider interface 250, and a data network interface 260. The AETA computer system 200 communicates with a user emotional state sensor data source computing system 290, hereafter referred to as a user computing system 290, via one or more data networks 280, such as a wide area network, e.g., the Internet, and a data network interface 260, to gather emotional state data for a user from various sensors and subsystems of the user computing system 290. The AETA computer system 200 further communicates with various other computing systems 282-288 to gather data to assist in training and runtime evaluation of the gathered emotion data, as will be described hereafter. Of particular note, the AETA computer system 200 operates in conjunction with a virtual environment provided by the virtual environment provider systems 282 and with which the user may interact via their user computing device 290. For example, the virtual environment provider systems 282 may be computer systems for presenting a virtual environment, such as virtual environment in the Metaverse or the like, and with which the user interacts via an avatar representation of the user in the virtual environment, as may be viewed/rendered by a virtual environment viewing/rendering engine 298 of the user computing device 290. The AETA computer system 200 operates to improve and enhance the user's experience within the virtual environment based on predicted current and/or future emotional states of the user given stimuli in the virtual environment.

In some illustrative embodiments, the AETA computer system 200 may operate, for example, as a cloud service employed by the user computing system 290 or the virtual environment provider systems 282 to augment and enhance the virtual environment representations for the various users of the virtual environment(s) provided by the virtual environment provider systems 282. As such the AETA computer system 200 may be remotely located and distributed, relative to the virtual environment provider systems 282, but may be accessible via data communications facilitated by the one or more data networks 280. It should be appreciated that while the AETA computer system 200 is shown as a separate computer system from the virtual environment provider systems 282, in some illustrative embodiments, the mechanisms of the AETA computer system 200 may be integrated with the virtual environment provider systems 282 to operate in conjunction with the mechanisms of the virtual environment provider systems 282 to present virtual environments to users via the user computing devices 290. That is, a user of a user computing device 290 may login or otherwise access the virtual environment provider systems 282 via the network 280 and gain access to the virtual environments provided by the virtual environment provider systems 282. As part of the virtual environment provider systems 282 operation, the virtual environment provider systems 282 may implement the improved computing tool and improved computing tool functionality of the AETA computer system 200 to personalize the virtual environment to the particular user, and specifically with regard to the learned associations of emotional state and stimuli, as discussed hereafter.

In some illustrative embodiments, aspects of the AETA system 200 may alternatively, or in addition, be implemented at the user computing device 290. For example, the AETA system 200 may train a machine learning computer model to specifically learn associations of emotional state and stimuli for a particular user, as well as build a user specific set of emotion and stimuli ontology data structures. These user specific instances, along with instances of emotion data collection 210 and virtual environment personalization engine 230 logic, may be deployed to the user computing device 290 which then operate local to the user computing device 290 and provide information to the virtual environment provider systems 282 for personalizing the virtual environments for the particular user. Thus, in such illustrative embodiments, the AETA computer system 200 may serve primarily as a machine learning/deep learning model configuration and training platform which then deploys instances to user computing devices 290 at which time the user computing devices 290 utilize the deployed instances to interact with the virtual environment provider systems 282 via the one or more data networks 280.

As shown in FIG. 2, the AETA computer system 200 comprises an emotion data collector 210 that operates to collect historical and runtime data representative of user emotions in response to stimuli. The emotion data collector 210 comprises a history data manager 212 and a runtime data manager 214. The history data manager 212 and runtime data manager 214 perform similar operations and comprise similar logic, but operate on different types of emotion data, specifically historical data which may include emotion labels and stimuli labels, and runtime data which does not include such labels. With regard to historical data, the historical data manager 212 may collect data from various sources that provide data representative of a user's physiological condition in response to various stimuli and this data may be labeled by the user and/or other authorized personnel to specify the emotions of the user and the stimuli being presented at same time that the user experiencing the emotion. For example, the emotion data may comprise brain wave patterns, heart rate information, perspiration levels, eye dilation information, breathing rate, facial expression information, body temperature, blood pressure, etc., and this combination of information may be associated with a labeled emotion, e.g., “afraid”, “happy”, “upset”, etc. Similarly, this information may be correlated with data representing one or more stimuli existing at substantially the same time in the environment of the user when the emotion data was recorded, and this stimuli data may be labeled with a label specifying what type of stimuli was present, e.g., a spider, a particular color, a particular other user, etc. This data may be collected for emotions/stimuli existing in the physical world and/or virtual environment, such that emotional states of the user may be determined whether the stimuli is physical or virtual.

The emotion data may be data collected from one or more users via sensor devices local to the users and which record and report data specifying measurements of various types to a user computing device 290, which in turn is provided to the AETA computer system 200 via the data network interface 260 and processed by the emotion data collector 210. The sensors may include wearable sensors 292 as well as physical environment sensors 296. The wearable sensors 292 may comprise various sensors that are worn on apparatus, clothing, e.g., smart clothing, or the like, where the sensors have contact with the user him/herself or are otherwise able to obtain measurement data from monitoring the user him/herself in close proximity to the user. For example, in some illustrative embodiments, the wearable sensors 292 may comprise a headset, an armband, a wristband, a smart watch, a smart glasses device, or any other device that has sensors for sensing one or more types of physiological conditions of the user. For example, the sensors may include sensors for detecting brain wave patterns, heart rate information, perspiration levels, eye dilation information, breathing rate, facial expression information, or any other physiological conditions and/or biometrics that may be indicative of emotional state of the user.

In some cases, the wearable sensors also provide sensors for sensing facial features, body language, micro language, and the like. For example, cameras or other image capturing devices may be associated with the apparatus, clothing, etc., that can capture images of the user's facial features, body language, lip motion, etc. This information may also be captured using physical environment sensors 296. The difference between physical environment sensors 296 and the wearable sensors 292 is that the physical environment sensors 296 may be remote from the user but present within the physical environment of the user such that the physical environment sensors 296 may capture information about the user and the environment within close proximity of the user. These environment sensors 296 may capture information about the user's body language, facial features, movements, as well as information about other entities, objects, or characteristics of the environment, e.g., what animals are present, which persons are present, colors, lighting conditions, temperature, etc.

The user computing device 290 further includes communications and event data engines 294 that provide mechanisms for the user of the user computing device 290 to communicate with other persons via electronic communications, e.g., emails, texts, etc., as well as via social networking systems, e.g., via posts, likes, etc. The event data engines provide electronic calendars or other data sources indicative of events that are being attended by the user. The information from the communications and event data engines 294 provide information about potential stimuli associated with user emotion data captured by the sensors 292, 296 and collected by the emotion data collector 210. For example, the emotion data collector 210 may communicate with the user computing device 290 to collect emotion data from the sensors 292, 296 as well as communications and events data from the communications and event data engines 294. The emotion data and the communications and event data may be timestamped such that the emotion data and communications/event data may be correlated to determine what emotion data was collected at substantially a same time as the communications/events occurring as specified in the communications/event data from engines 294. In this way, the emotion data may be correlated with physical environment stimuli to determine associations between emotion data and physical environment stimuli that cause corresponding emotions.

Similarly, virtual environment data may be gathered from the virtual environment viewer/rendering engine 298 and/or virtual environment provider systems 282. Thus, where the communications and event data engines 294 and physical environment sensors 296 may provide information indicating stimuli causing the user's emotional state represented in the emotion data, the virtual environment viewer/rendering engine 298 and virtual environment provider systems 282 provides information specifying the stimuli present in the virtual environment that may cause an emotional state of the user as represented in the emotion data gathered from sensors 292, 296. Thus, for example, virtual objects, other avatars of other users, environmental conditions virtually represented, e.g., weather effects, lighting conditions, colors utilized, etc., may all be collected information that may be indicative of stimuli that may elicit emotional states of the user as may be represented by emotion data collected from the sensors 292, 298.

As shown in FIG. 2, other data sources that may provide data for emotion classification/prediction may include social networking data systems 284, location data systems 286, and event data systems 288, for example. The social networking data systems 284 provide data about associations between users and other users via social networking systems, as well as the negative/positive nature of these associations, as may be determined from evaluating communications between these users, likes/dislikes, blocking of user posts, selection of users as “friends”, or any other information indicative of positive/negative relationships between users. In some illustrative embodiments, natural language processing of communications, posts, and the like, may be utilized to perform sentiment analysis of communications between users and thereby identify emotional views of one user toward another user.

The location data systems 286 provide location information for the physical location of the user when the user's computing device 290 senses emotion data via the sensors 292 and/or 296. That is, the location data systems 286 may comprise global positioning systems (GPS), cellular triangulation systems, or any other mechanism for identifying the physical location of a user in the physical environment. This location information may include not only coordinates, but also information about the particular location corresponding to those coordinates, e.g., what type of establishment is present at the location, what services/products the establishment provides if any, or other characteristics of the location that may be indicative of stimuli that may evoke emotional states in the user that may be represented in the emotion data collected form the sensors 292, 296.

Thus, various types of data representing physiological, biometric, and facial/body language features, which are representative of an emotional state of a user may be collected and referred to generally as emotion data. In addition, various types of data representing the environments, physical and/or virtual, in which the user is present (physically or virtually through an avatar in the virtual environment), are collected to represent the various stimuli that are experienced by the user at the time that the emotion data is collected. This stimuli sets forth a context for the emotion data and thus, may be referred to herein as stimuli context data.

During training of a machine learning or deep learning computer model in 240, this emotion data and stimuli context data may be labeled by the user and/or other authorized personnel and stored in a historical database 272 associated with a user registry 270 entry corresponding to the user. This historical database 272 may be built up over time to include labeled emotion data and labeled stimuli context data for the user, with historical databases 272 being generated for a plurality of users. The historical database 272 may be input to an emotion classifier/predictor engine 220 to train one or more machine learning (ML)/deep learning (DL) models 222 using ML/DL model training engine 240 and the machine learning training algorithms configured in the ML/DL model training engine 240. That is, the historical data may be used as training data that is input to the ML/DL model(s) 222 which generate a classification/prediction and this classification/prediction is compared to the labels for the emotion data. Based on a determined error in the generated classification/prediction relative to the label, and the identification of the contributing nodes of the ML/DL model that contributed more/less strongly to the classification/prediction, weights of nodes may be adjusted so as to minimize the error in the classification/prediction, e.g., through a linear regression or other machine learning training algorithm. This may be done in an iterative manner until the determined error is equal to or less than a predetermined threshold level of error or until a predetermined number of iterations, or epochs, have occurred.

The training causes the ML/DL model(s) 222 to learn associations between patterns in input data and corresponding classifications/predictions of an emotional state of the user. For example, a ML/DL model may be trained on input data representing brain wave patterns and predict, based on the brain wave patterns what the emotional state of the user is and the correlation of that emotional state with one or more stimuli contexts. Similarly, a ML/DL model may be trained on heart rate data patterns and predict, based on the heart rate data patterns what the emotional state of the user is for a given stimuli context. A single ML/DL model 222 may process each of these different types of input data and generate a prediction of emotional state and correlate that emotional state with the given stimuli context.

Alternatively, multiple ML/DL models 222 may be trained, each operating on one or a subset of the emotion data as inputs and generating a corresponding prediction of emotional state for a given stimuli context, such as in the case of an ensemble of ML/DL models 222. In some illustrative embodiments, one ML/DL model 222 may generate a prediction that is fed as additional input to a subsequent ML/DL model 222 and upon which the subsequent ML/DL model 222 operates to generate its own prediction of emotional state for the given stimuli context, and so on, in a pipeline type manner. A meta-model engine 224 may operate to combine the outputs of the various ML/DL models 222 to generate a final emotion classification/prediction for a given stimuli context. The meta-model engine 224 may be likewise trained through machine learning processes to weight the outputs of the various ML/DL models 222 according to learned weightings so as to more or less rely on outputs of different classifications/predictions of the ML/DL models 222 for providing an accurate classification/prediction of user emotional state for particular stimuli contexts.

The learned associations of stimuli context and corresponding emotional states may be used to build an emotion/stimuli ontology data structure (ontology data structure) 274 for the user. The ontology data structure 274 correlates different stimuli contexts with corresponding emotions of the user. The ontology data structure 274 may be initially a generalized ontology data structure that is generated across a plurality of users, and which is then updated/modified based on user specific classifications/predictions of emotional state to represent how the particular user responds to the particular stimuli context. Thus, for example, the ontology data structure 274 may represent that, for this user, the user has an emotional state of “afraid” and “apprehensive” with regard to a stimuli context comprising a “spider”, where the ontology data structure 274 or other linked data structures may specify the characteristics of the emotional states and the stimuli context, e.g., “afraid” for this user corresponds to brain wave pattern X, heart rate pattern Y, blood pressure Z, etc. and the spider has characteristics of “animal type: arachnid”, “number of legs: 8”, number of eyes: many”, “size: small, medium”, etc. These characteristics may be used by the virtual environment personalization engine 230 to determine how to modify an environmental theme of a virtual environment to achieve a desired emotional state of the user. For example, if an environment contains an object that is not in the emotion/stimuli ontology 274 for the user, then a closest stimuli in the ontology 274 having similar characteristics to the characteristics of the object tin the environment may be identified through a matching operation to identify a similar stimuli context and corresponding emotional state of the user that is likely to be present or to occur given the object in the environment. For example, assume that there is an animal in the virtual environment that is not a spider, but resembles a spider in that it has similar characteristics, then the most likely emotional response of the user will be to be “afraid” or “apprehensive”.

Thus, through the operation of the emotion data collector 210, the user registry 270, the ML/DL model training engine 240, and emotion classifier/predictor engine 220, using data communications via the data network interface 260 to collect emotion and stimuli context data from various sensors and data sources, one or more ML/DL models 222 are trained to predict an emotional state of a user given a stimuli context based on the sensor data, and an emotion/stimuli context ontology 274 is generated for the user in the user registry 270. During runtime operation, the trained ML/DL models 222 and meta-model 224 may operate on runtime sensor data and stimuli context data to classify/predict the user's current and/or future emotional state given the stimuli context and sensor data input. This may correlate certain patterns of emotion data, given a stimuli context, with an emotional state. This emotional state may then be used along with the stimuli context to identify from the ontology 274 for the user, the likely cause of the emotional state, e.g., the user is afraid of spiders and thus, if the stimuli context includes an object resembling a spider, and the user's emotional state is predicted to be “afraid”, then it is most likely that the user's emotional state is due to the presence of an object resembling a spider.

Thus, the runtime data manager 214 of the emotion data collector 210 may collect runtime data from the user computing device 290, such as sensor data from sensors 292, 296, and use that runtime data to predict the emotional state of the user via the emotion classifier/predictor engine 220. The cause of this emotional state may be determined by the virtual environment personalization engine 230 using the emotion/stimuli ontology 274. That is, the stimuli context may have a plurality of potential stimuli present in the context that may be the potential cause of a classified/predicted emotional state of the user. The virtual environment personalization engine 230 may be trained on the historical data of the user, in a similar manner as discussed above for emotion classifier/predictor engine 220, but to predict environmental themes, or stimuli, associated with emotional states given a stimuli context and emotional state, as well as the emotion/stimuli ontology 274. That is given a stimuli context and emotional state prediction/classification from the emotion classifier/predictor engine 220, the virtual environment personalization engine 230 comprises one or more ML/DL models 232 that predict what elements of an environment, i.e., the stimuli context, are associated with the emotion state and predicts which one or ones are most likely to be the cause of the emotional state. For example, if the environment includes a chair, a table, and a spider, and the emotional state is “afraid”, then the virtual environment personalization engine 230 may have trained ML/DL models 232 that evaluate this input data, the emotional state data, and the emotion/stimuli ontology 274, to determine that the most likely cause of the emotional state of “afraid” is not the chair or the table, but rather the spider.

This information may drive a change or modification of an environmental theme by theme selection/modification engine 234. That is, the theme selection/modification engine 234 may determine a desired emotional state of users that is to be elicited by the location of the user's avatar in the virtual environment. If that desired emotional state does not match the current/predicted emotional state of the user, then an environment theme selection/modification may be performed to modify the virtual environment to elicit the desired emotional state of the user. This may involve evaluating the emotion/stimuli ontology data structure 274 for the user to identify stimuli that elicit the desired emotional state from this particular user.

The desired emotional state may be determined from the virtual environment provider systems 282 based on the user's avatar's current location within the virtual environment. That is, the virtual environment provider systems 282 may have metadata associated with locations within the virtual environment that specify the desired emotional states for the various locations. Thus, the desired emotional state may be obtained from the virtual environment provide systems 282 and compared to the emotional state predicted for the user. If there is a discrepancy between the two, then the virtual environment personalization engine 230 may select an environment theme or modification of the environment theme that is likely to elicit the desired emotional state from the user.

In the context of environments, a “theme” is used to create a cohesive and immersive experience for the user or avator by tying together different elements such as colors, patterns, textures, and décor, etc. For example, a beach-themed environment might include pastel blues and greens, seashells, and images of waves and sand, while a jungle-themed environment might include shades of green, exotic plants, and animal prints. With the illustrative embodiments, the concept of “themes” is related to creating customizable and adaptable environments that can be changed according to the analyzed result. By defining different themes that incorporate specific design elements and decor, the avator can easily see between different atmospheres or moods within the same metaverse. This could be achieved through the use of technology or modular design elements that can be easily interchanged.

The selection of an environment theme or modification to a theme may be specific to the predicted cause of the predicted emotional state of the user generated by the virtual environment personalization engine 230, e.g., the particular elements of the stimuli context that are likely the cause of the emotional state as determined using the emotion/stimuli ontology 274. There may be a plurality of different predefined themes or even individual stimuli that may be represented in the virtual environment, and from which the theme selection/modification engine 234 may select based on the emotion/stimuli ontology 274 of the user. Based on the emotion/stimuli ontology 274, one or more matching stimuli context that matches the desired emotional state may be selected. From this subset of matching stimuli contexts, a stimuli context may be selected that has characteristics that are determined to be most similar to the stimuli context that is determined to be the likely cause of the emotional state of the user as predicted by the emotion classifier/predictor engine 220 and correlated with a causal stimuli context by the virtual environment personalization engine 230.

For example, assume that the predicted emotional state of a user is “afraid”, but the desired emotional state is “excited”. Also assume that the virtual environment personalization engine 230 determines that the most likely cause of the “afraid” state is the presence of a spider in the virtual environment. The theme selection/modification engine 234 determines that the desired and predicted emotional states differ from each other. Thus, the theme selection/modification engine 234 analyzes the emotion/stimuli ontology 274 for one or more stimuli contexts that elicit from the user an “excited” state. This subset of stimuli contexts are then further analyzed to find a closest match of stimuli context characteristics to the causal stimuli context characteristics of the spider. As a result, an environment theme, replacement stimuli, or the like, may be selected for modifying the virtual environment. For example, the spider may be replaced with a mouse, dog, or other entity that either is associated with the desired emotional state, or does not cause the predicted emotional state of the user. In some cases, if a suitable environmental theme or stimuli cannot be identified for selection/modification, then the causal stimuli context may be eliminated from the virtual environment, e.g., the spider is simply removed from the virtual environment.

As another example, consider a scenario in which the user's avatar is present in a virtual environment location of a virtual restaurant in the Metaverse. However, sensor data gathered from the user's wearable sensors and/or environment sensors, which are input to the trained emotion classifier/predictor engine 220 indicate a predicted emotional state of the user to be “upset” or “sad”. Assume that the provider of the virtual environment has associated with the restaurant location, a desired emotional state of “happy” or “satisfied”. Thus, there is a discrepancy between the current emotional state of the user and the desired emotional state of the user.

Assume also that through operation of the virtual environment personalization engine 230, operating on the emotion/stimuli ontology 274, that the likely cause of the user's current emotional state is a lack of items on a menu of the restaurant that the user likes. That is, the menu is the likely source of the “upset” or “sad” emotional state of the user. As a result, the theme selection/modification engine 234 may look to the emotion/stimuli ontology 274 to identify stimuli contexts that are associated with the desired emotional state of “happy” or “satisfied”. This subset of stimuli contexts are then further analyzed to identify stimuli that have characteristics most resembling the determined likely cause of the user's current emotional state, i.e., the menu and/or items on the menu. The most similar stimuli or stimuli context may then be used to modify or replace the causal stimuli context in hopes of modifying the user's emotional state to be that of the desired emotional state. This process may be performed repeatedly, keeping track of which selections/modifications were previously attempted, until the desired emotional state is achieved.

The theme selections/modifications generated by the theme selection/modification engine 234 are communicated to the virtual environment provider systems 282 via the virtual environment provider interface 250. The theme selections/modifications are communicated with the virtual environment provider systems 282 which modify the presentation of virtual environments to the user via the user computing device 290 and virtual environment viewer/rendering engine 298. It should be appreciated that this modification is specific to the user of user computing device 290 and other users of other user devices in the same location within the virtual environment may perceive other versions of the location that do not include the environmental theme/modifications selected for the user of user computing device 290. Thus, each individual user may have their own personalized representation of the same location within the virtual environment so as to elicit desirable emotional states or avoid undesirable emotional states and make the virtual environment more immersive to the specific user.

In some cases, the virtual environment personalization engine 230 may further include value added options that may be determined by the theme selection/modification engine 234 based on the discrepancy between the predicted and desired emotional states. For example, various services and virtual products may be offered to the user based on their predicted emotional state and the desired emotional state so as to attempt to bring the user's predicted emotional state in line with the desired emotional state. For example, if the user is not pleased with a variety of product offerings at a virtual vendor, a value added offering may provide alternative virtual products that are determined from the user's emotion/stimuli context ontology 274 that are associated with the desired emotional state, e.g., the user does not like a particular type of food so the food offerings may be modified to provide a different type of food that the user likes as indicated in their ontology 274. Other types of value added options may be provided without departing from the spirit and scope of the present invention. For example, other value added options may include changing the order of a menu according to the predicted emotional state or adding additional items in a menu, recommending relaxing virtual activities such as meditation or yoga to help the user feel calmer and more centered if the predicted emotional state is “anxious or stressed”, offering advertisements for travel destinations or recreational activities if the predicted emotional state is “happy and relaxed”, or the like.

In view of the above description, it is clear that the present invention provides an improved computing tool and improved computing tool functionality for personalizing virtual environments of user avatars based on a machine learning learned association between user emotional states and stimuli contexts within the physical world and the virtual world. The virtual environments of the virtual world may be modified based on these learned associations so as to elicit from the user an emotional state that is desired by the virtual environment provider for the specific virtual location of the user's avatar within the virtualized environment. Thus, if a location is to elicit fear in the user, then the virtual environment may be adapted through selection of virtual environment themes and/or stimuli contexts that specifically cause that user to have an “afraid” emotional state. Thus, for one user this may involve having virtual spiders present in the virtual environment, whereas for a different user, this may require that the environment have snakes or bugs present in the virtual environment. In some cases, value added services may be provided to the user based on determined discrepancies between predicted and desired emotional states so as to give the user options to modify their virtual environment to make the virtual environment more amenable to the user. All of these mechanisms operate to provide a more immersive experience of the virtual environment for the user and personalize the virtual environment to the particular user.

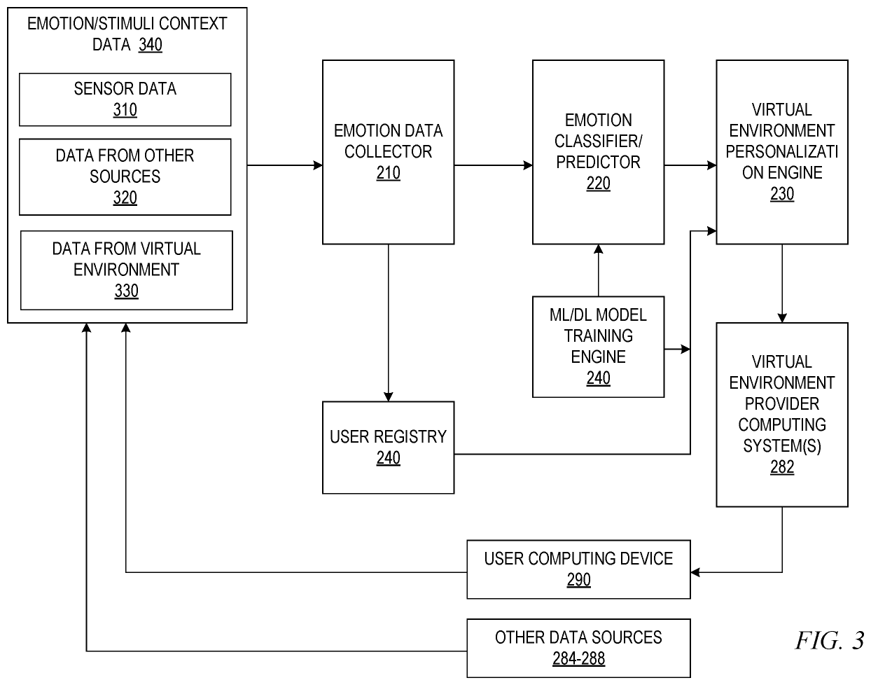

FIG. 3 is an example data flow diagram for an AETA computing system in accordance with one illustrative embodiment. The operation shown by the data flow of FIG. 3 is the same as described above with regard to FIG. 2, but is represented as a flow of data from one component to another in the AETA computing system. As shown in FIG. 3, emotion/stimuli context data 340 is obtained from the user computing device 290 and other data sources 282-288. That is, the sensor data 310 may be obtained from wearable and environmental sensors of the user device 290, data from the virtual environment 330 may be obtained from the virtual environment provider computing systems 282, and other data 320 indicative of stimuli contexts, e.g., location data and the like, may be obtained from other data source computing systems 284-288, for example. The emotion/stimuli context data 340 is collected by the emotion data collector 210 and is used during training to train one or more ML/DL computer models, and is used during runtime to predict the user's emotional state and select environmental themes/modifications for driving modifications of a virtual environment of a user's avatar. During training operations the ML/DL model training engine 240 trains ML/DL models in the emotion classifier/predictor engine 220 and the virtual environment personalization engine 230 using historical data from the user registry 270.

During runtime operations, the emotion classifier/predictor engine 220 may operate on the emotion/stimuli context data 340 to predict a current and/or future emotional state of the user given a stimuli context and input emotion data. The predicted emotional state of the user may be input to the virtual environment personalization engine 230 along with the emotion/stimuli ontology data structure from the user registry 270 to predict a causal stimuli context of the emotional state of the user, a discrepancy between the predicted emotional state and a desired emotional state, and an environment theme selection/modification to address the discrepancy between the predicted emotional state and the desired emotional state. The environmental theme selection/modification is then output to the virtual environment provider computing system 282 to modify the presentation of a virtual environment to the user of the user computing device 290. The operation may be repeated, such as in response to a location change within the virtual environment or other triggering condition desirable to the particular implementation.

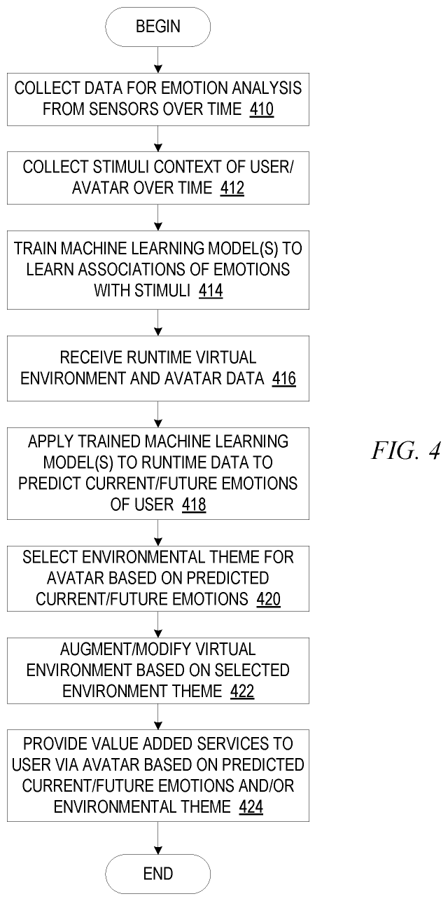

FIGS. 4-6 present flowcharts outlining example operations of elements of the present invention with regard to one or more illustrative embodiments. It should be appreciated that the operations outlined in FIGS. 4-6 are specifically performed automatically by an improved computer tool of the illustrative embodiments and are not intended to be, and cannot practically be, performed by human beings either as mental processes or by organizing human activity. To the contrary, while human beings may, in some cases, initiate the performance of the operations set forth in FIGS. 4-6, and may, in some cases, make use of the results generated as a consequence of the operations set forth in FIGS. 4-6, the operations in FIGS. 4-6 themselves are specifically performed by the improved computing tool in an automated manner.

FIG. 4 is a flowchart outlining an example operation of an AETA computing system in accordance with one illustrative embodiment. As shown in FIG. 4, the operation starts by collecting emotion data for emotion analysis from sensors over time (step 410). This may result in the historical data that is then labeled for use as training/testing data to train/test one or more ML/DL models, as discussed previously. The stimuli context data is also collected over time, both with regard to the physical environment and the virtual environment, and labeled as well so as to provide training/testing data for machine learning training of one or more ML/DL models (step 412). It should be appreciated that this data may be timestamped so as to allow for correlating the stimuli context data with the emotion data and thereby draw associations between stimuli and emotion data. One or more ML/DL models are trained based on this historical data from steps 410 and 420 so as to learn associations between emotions and stimuli contexts (step 414).

Having trained the one or more ML/DL models, runtime user emotional data, virtual environment data, and avatar data are received (step 416) and input to the trained ML/DL model(s). the trained ML/DL model(s) are applied to the runtime data to predict current/future emotions of the user given the stimuli context of the virtual environment (step 418). Based on the predicted current/future emotions of the user, and potentially desired emotional states of the user, environmental themes/modifications to virtual environments are selected for the user's avatar (step 420). The virtual environment is then augmented/modified based on the selected environmental theme/modification (step 422). Value added services may also be provided to the user via their avatar base don the predicted current/future emotions and/or selected environmental theme/modification (step 424). The operation then terminates.

It should be appreciated that while the flowchart terminates, the operation of the flowchart, during runtime operation, may be repeated with regard to steps 416-424 in response to triggering conditions, e.g., a change of location of the user's avatar within the virtual environment, or the like. Moreover, the operation of FIG. 4 may be repeated for different users.

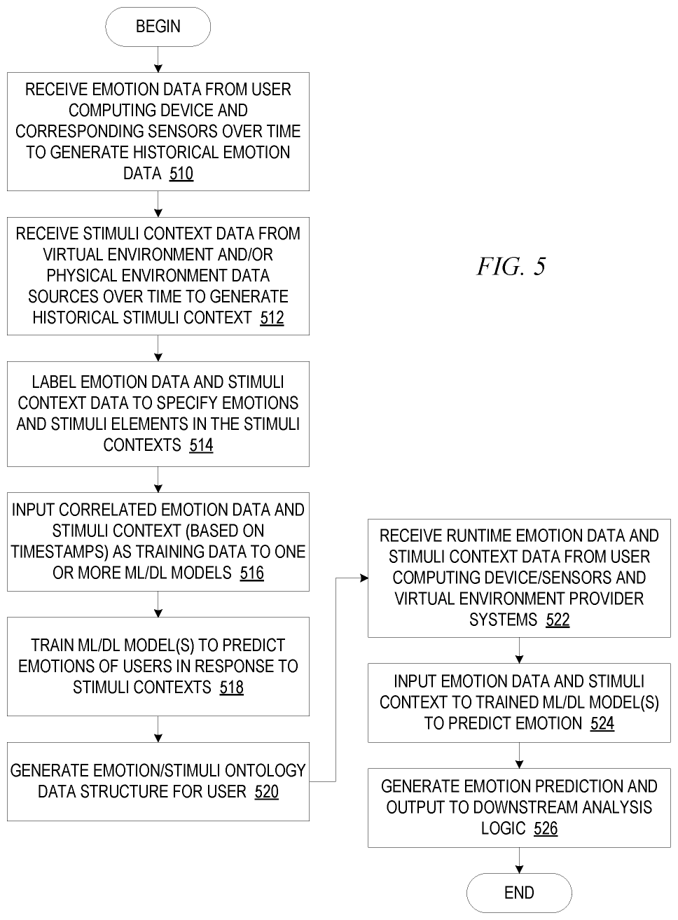

FIG. 5 is a flowchart outlining an example operation of an emotion classifier/predictor engine of an AETA computing system in accordance with one illustrative embodiment. The operation outlined in FIG. 5 may be implemented, for example, as part of operations 410-418 of FIG. 4, for example. The operation outlined in FIG. 5 trains one or more ML/DL computer models and uses the trained ML/DL computer models to perform a runtime evaluation of emotion data based on the learning of associations between patterns in emotion data and user emotions given stimuli contexts.

As shown in FIG. 5, the operation starts by receiving emotion data from sensors, wearable and environmental, and a corresponding user computing device to which the sensors report their data. This emotion data is received over time to thereby generate historical emotion data (step 510). Similarly, stimuli context data is received over time from one or more virtual and/or physical environment data sources to thereby generate historical stimuli context data (step 512). The emotion data and stimuli context data are labeled to identify the emotions and the elements of the stimuli context (step 514). For example, a user may specify what emotions they were feeling when the emotion data was sensed and recorded, thereby correlating the sensor data, such as brainwave patterns, heart rate, blood pressure, eye dilation, lip, body, and other micro language data, etc., with emotions. Similarly, a user may specify what elements were in the stimuli context, e.g., what objects, entities, and other characteristics of the environment were present such that they may serve as stimuli giving rise to the labeled emotion.

The labeled emotion data and stimuli context data are correlated by timestamps and input as training data to one or more ML/DL models (step 516) and a machine learning training process is performed on the ML/DL models to train the models to predict emotions of users in response to stimuli contexts (step 518). In this way, a trained set of one or more ML/DL models are generated that can, given input emotion and context data, predict a user's emotional state, which may be a current emotional state or a future emotional state. An emotions/stimuli ontology data structure for the user is generated that correlates emotional states with stimuli contexts for the user, and which is stored in association with the user's registry entry (step 520).

Thereafter, runtime (unlabeled) emotion data and stimuli context data are received from the user computing device/sensors and virtual environment provider systems (step 522). That is, the stimuli context data is the stimuli context of the virtual environment that the user's avatar is, or will be, present in. Thus, while during training the ML/DL models may learn associations from both physical and virtual environment stimuli contexts, during runtime, the stimuli context data that is input is the stimuli context data from the virtual environment of the user's avatar.

The received runtime (unlabeled) emotion data an stimuli context are input to the trained ML/DL models to predict at least one emotion of the user given the stimuli context (step 524). The emotion prediction is then generated and output to further downstream analysis logic (step 526). The operation then terminates.

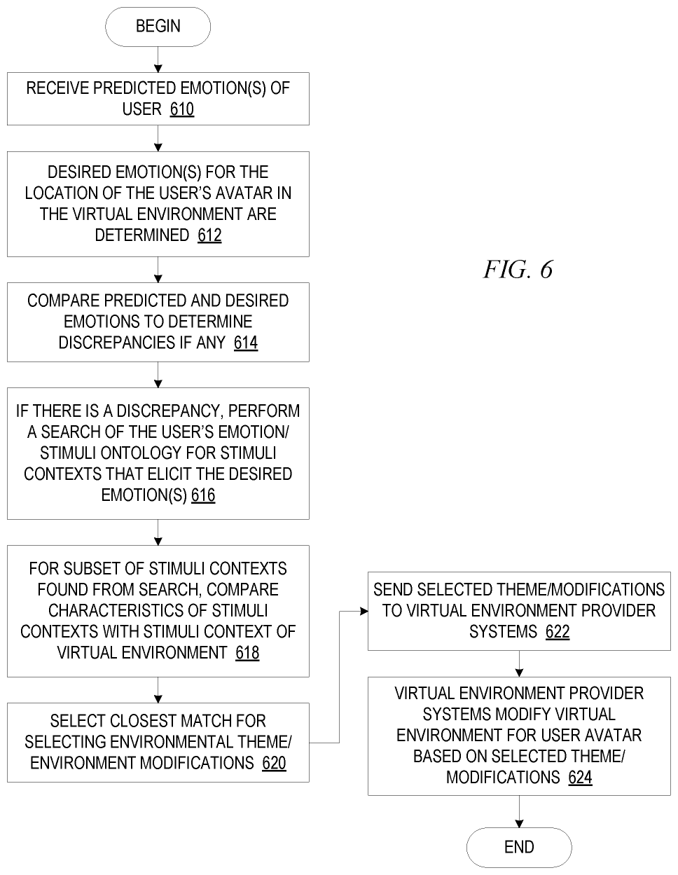

FIG. 6 is a flowchart outlining an example operation of a virtual environment personalization engine of an AETA computing system in accordance with one illustrative embodiment. The operation outlined in FIG. 6 may be implemented, for example, as part of operations 420-422 of FIG. 4, for example. As shown in FIG. 6, the operation starts by receiving the predicted emotion(s) of the user given the emotion data and stimuli context data from the virtual environment (step 610). Desired emotions(s) for the location of the user's avatar in the virtual environment are determined (step 612). The predicted and desired emotions are compared to determine any discrepancies, if any (step 614). If there is a discrepancy, a search of the user's emotion/stimuli ontology is performed for stimuli contexts that elicit the desired emotion(s) (step 616). For the subset of stimuli contexts found from the search, the characteristics of the stimuli contexts are compared with the stimuli context of virtual environment (step 618).

A closest match for is selected for selecting an environment theme/environment modification to be made to the virtual environment to elicit the desired emotional state from the user (step 620). A corresponding selected theme/environment modification is generated and sent to a virtual environment provider system (step 622). The virtual environment provider systems modify the virtual environment for the user avatar based on the selected theme/modifications (step 624). The operation then terminates.

While the above description provides an overview of software, hardware, and the configuration of such software, hardware, and such to implement various “engines”, it should be appreciated that any references to generic computing hardware is intended to refer to merely the hardware itself in cases where the hardware is not modified. However, even if, in some embodiments, generic computing hardware is used as a basis, the invention is not in the generic computing hardware, but rather the specifically configured software and hardware mechanisms that, only through such specific configuration, permit the described inventive computer tool functionalities to be realized. That is, for a computing tool to provide improved or inventive computing tool functionality, the computing tool relies on a combination of hardware and software that together define the improved computing tool functionality, unless new hardware is specifically described that hard wires this specific configuration into a new arrangement of circuitry. Hence, even in embodiments where the “engines” are implemented in software executing on computer hardware which configures that computer hardware to perform the particular improved computing tool functionalities of the embodiment, the embodiment is describing an improved computer functionality and improved computing tool and not an abstract idea for which computers are merely used as a tool. The embodiments described herein are not directed to any abstract idea of the invention, but rather to a practical application of an improved computing tool and improved computing tool functionality.

The description of the present invention has been presented for purposes of illustration and description, and is not intended to be exhaustive or limited to the invention in the form disclosed. Many modifications and variations will be apparent to those of ordinary skill in the art without departing from the scope and spirit of the described embodiments. The embodiment was chosen and described in order to best explain the principles of the invention, the practical application, and to enable others of ordinary skill in the art to understand the invention for various embodiments with various modifications as are suited to the particular use contemplated. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, in a data processing system, for personalizing a computer generated virtual environment, the method comprising:
collecting, from one or more sensors associated with a user, emotion data representing physiological conditions of the user in response to stimuli;
collecting, from one or more data source computing systems, stimuli context data and correlating the stimuli context data with the emotion data;
training, via a machine learning training process, one or more machine learning computer models based on the emotion data and correlated stimuli context data to thereby generate one or more trained machine learning computer models that are trained to predict an emotion of the user from patterns of input data;
receiving runtime emotion data from the one or more sensors associated with the user;
receiving runtime stimuli context data from a virtual environment provider computing system for the computer generated virtual environment; and
generating, by the one or more trained machine learning computer models, a predicted emotion of the user based on the runtime emotion data and runtime stimuli context data which are input to the one or more trained machine learning computer models.

2. The method of claim 1, further comprising modifying a virtual environment theme of the computer generated virtual environment based on the predicted emotion of the user.

3. The method of claim 2, wherein modifying the virtual environment theme of the computer generated virtual environment based on the predicted emotion of the user comprises at least one of modifying the virtual environment theme to elicit an intended emotion from the user that is different from the predicted emotion of the user, or modifying the virtual environment theme to elicit the predicted emotion of the user from the user.

4. The method of claim 1, wherein the correlated stimuli context data comprises at least one of visual stimuli, auditory stimuli, environmental stimuli, social stimuli, object stimuli, or contextual stimuli as specified in a stimuli ontology data structure, and wherein the stimuli ontology data structure is input to the one or more machine learning computer models to train the one or more machine learning computer models to predict the emotion of the user from patterns of input data.

5. The method of claim 1, wherein the emotion data comprises an emotional state ontology data structure specifying a plurality of predefined emotional states of a user, and wherein portions of the emotion data are correlated with corresponding ones of predefined emotional states in the emotional state ontology data structure, and wherein the emotional state ontology data structure is input to the one or more machine learning computer models to train the one or more machine learning computer models to predict the emotion of the user from patterns of input data.

6. The method of claim 1, wherein the emotion data is collected from the one or more sensors associated with a user in response to stimuli present in a physical environment, and wherein the stimuli context data represents the stimuli present in the physical environment, such that the one or more machine learning computer models learn associations of input patterns of emotional responses of the user to stimuli in the physical environment, and applies the learning to virtual stimuli in the virtual environment.

7. The method of claim 1, wherein the emotion data is collected from the one or more sensors associated with a user in response to virtual stimuli present in a virtual world environment, and wherein the stimuli context data represents the virtual stimuli present in the virtual world environment, such that the one or more machine learning computer models learn associations of input patterns of emotional responses of the user to virtual stimuli in the virtual world environment, and applies the learning to virtual stimuli in the virtual environment.

8. The method of claim 1, wherein the emotion data comprises at least one of brain wave pattern data, heart rate pattern data, perspiration level data, eye dilation data, breathing rate data, facial expression data, body temperature data, or blood pressure data.

9. The method of claim 1, wherein the one or more sensors comprise at least one of a user wearable sensor or a sensor physically positioned in a physical environment occupied by the user, to monitor the user while the user occupies the physical environment.

10. The method of claim 1, wherein the stimuli context data comprises at least one of:
physical environment stimuli context data, collected from the one or more sensors, specifying objects, entities, or physical conditions of a physical environment in which the user occupies at a substantially same time as the emotion data is collected;
social networking stimuli context data, from one or more social networking computing systems, specifying relationships between the user and other entities present in the physical environment in which the user occupies at a substantially same time as the emotion data is collected;
event stimuli context data, from one or more event data source computing systems, specifying events occurring in the physical environment in which the user occupies at a substantially same time as the emotion data is collected; or
location stimuli context data, from one or more location services computing systems, specifying characteristics of a location of the user corresponding to the physical environment in which the user occupies at a substantially same time as the emotion data is collected.

11. A computer program product comprising a computer readable storage medium having a computer readable program stored therein, wherein the computer readable program, when executed on a data processing system, causes the data processing system to:
collect, from one or more sensors associated with a user, emotion data representing physiological conditions of the user in response to stimuli;
collect, from one or more data source computing systems, stimuli context data and correlating the stimuli context data with the emotion data;
train, via a machine learning training process, one or more machine learning computer models based on the emotion data and correlated stimuli context data to thereby generate one or more trained machine learning computer models that are trained to predict an emotion of the user from patterns of input data;
receive runtime emotion data from the one or more sensors associated with the user;
receive runtime stimuli context data from a virtual environment provider computing system for the computer generated virtual environment; and
generate, by the one or more trained machine learning computer models, a predicted emotion of the user based on the runtime emotion data and the runtime stimuli context data which are input to the one or more trained machine learning computer models.

12. The computer program product of claim 11, wherein the computer readable program further causes the data processing system to modify a virtual environment theme of the computer generated virtual environment based on the predicted emotion of the user.

13. The computer program product of claim 12, wherein modifying the virtual environment theme of the computer generated virtual environment based on the predicted emotion of the user comprises at least one of modifying the virtual environment theme to elicit an intended emotion from the user that is different from the predicted emotion of the user, or modifying the virtual environment theme to elicit the predicted emotion of the user from the user.

14. The computer program product of claim 11, wherein the correlated stimuli context data comprises at least one of visual stimuli, auditory stimuli, environmental stimuli, social stimuli, object stimuli, or contextual stimuli as specified in a stimuli ontology data structure, and wherein the stimuli ontology data structure is input to the one or more machine learning computer models to train the one or more machine learning computer models to predict the emotion of the user from patterns of input data.

15. The computer program product of claim 11, wherein the emotion data comprises an emotional state ontology data structure specifying a plurality of predefined emotional states of a user, and wherein portions of the emotion data are correlated with corresponding ones of predefined emotional states in the emotional state ontology data structure, and wherein the emotional state ontology data structure is input to the one or more machine learning computer models to train the one or more machine learning computer models to predict the emotion of the user from patterns of input data.

16. The computer program product of claim 11, wherein the emotion data is collected from the one or more sensors associated with a user in response to stimuli present in a physical environment, and wherein the stimuli context data represents the stimuli present in the physical environment, such that the one or more machine learning computer models learn associations of input patterns of emotional responses of the user to stimuli in the physical environment, and applies the learning to virtual stimuli in the virtual environment.

17. The computer program product of claim 11, wherein the emotion data is collected from the one or more sensors associated with a user in response to virtual stimuli present in a virtual world environment, and wherein the stimuli context data represents the virtual stimuli present in the virtual world environment, such that the one or more machine learning computer models learn associations of input patterns of emotional responses of the user to virtual stimuli in the virtual world environment, and applies the learning to virtual stimuli in the virtual environment.

18. The computer program product of claim 11, wherein the emotion data comprises at least one of brain wave pattern data, heart rate pattern data, perspiration level data, eye dilation data, breathing rate data, facial expression data, body temperature data, or blood pressure data.

19. The computer program product of claim 11, wherein the one or more sensors comprise at least one of a user wearable sensor or a sensor physically positioned in a physical environment occupied by the user, to monitor the user while the user occupies the physical environment.

20. An apparatus comprising:
at least one processor; and
at least one memory coupled to the at least one processor, wherein the at least one memory comprises instructions which, when executed by the at least one processor, cause the at least one processor to:
collect, from one or more sensors associated with a user, emotion data representing physiological conditions of the user in response to stimuli;
collect, from one or more data source computing systems, stimuli context data and correlating the stimuli context data with the emotion data;
train, via a machine learning training process, one or more machine learning computer models based on the emotion data and correlated stimuli context data to thereby generate one or more trained machine learning computer models that are trained to predict an emotion of the user from patterns of input data;
receive runtime emotion data from the one or more sensors associated with the user;
receive runtime stimuli context data from a virtual environment provider computing system for the computer generated virtual environment; and
generate, by the one or more trained machine learning computer models, a predicted emotion of the user based on the runtime emotion data and the runtime stimuli context data which are input to the one or more trained machine learning computer models.
